(19) United States
(12) Patent Application Publication
(10) Pub. No.: US 2012/0143014 A1
(43) Pub. Date: Jun. 7, 2012

(54) HEALTHCARE SYSTEM AND HEALTHCARE METHOD

(75) Inventors: Dong Woo KIM, Yongin-si (KR); Jong Rip LEE, Bucheon-si (KR); Se Un KIM, Seoul (KR); Sung Hwa LEE, Anyang-Si (KR)

(73) Assignee: SAMSUNG ELECTRONICS CO., LTD., Suwon-si (KR)

(21) Appl. No.: 13/301,531

(22) Filed: Nov. 21, 2011

(30) Foreign Application Priority Data
Dec. 7, 2010 (KR) 10-2010-0124106

(51) Int. Cl. A61B 5/00

(52) U.S. Cl. 600/300

(57) ABSTRACT

A healthcare system is provided. The healthcare system includes a server configured to receive user information and obtain healthcare items based on the information, a gateway configured to receive the healthcare items, and a vital information measurement sensor configured to measure vital information of a user.

### CROSS-REFERENCE TO RELATED APPLICATION(S)

This application claims the benefit under 35 U.S.C. §119(a) of Korean Patent Application No. 10-2010-0124106, filed on Dec. 7, 2010 in the Korean Intellectual Property Office, the entire disclosure of which is incorporated herein by reference for all purposes.

### BACKGROUND

1. Field

The following disclosure relates to a healthcare system and a healthcare method.

2. Description of the Related Art

A remote medical service system often referred to as a home healthcare system or a U-healthcare system may remotely measure vital information of a patient using various measurement apparatuses. The remote medical service system reduces visits to a hospital because the patient is able to remotely receive medical services, for example, at home, based on the measurement result from the remote medical service system.

In the remote healthcare system, a gateway is a patient terminal that may be used to collect vital information of a patient using various measurement apparatuses and to transmit the vital information to a hospital.

A typical method using a typical home healthcare system includes measuring all measurable data of a patient using a gateway and extracting data related to treatment of a patient from the data.

In such a method, all vital information of a patient is measured, collected, and transmitted to a gateway regardless of the disease that the patient suffers from. In this example, in order to obtain a small amount of information related to treatment of a patient in relation to the measured and collected information, a large amount of unrelated information needs to be measured. In addition, if a measurement method is invasive or restricted, the patient may be inconvenienced when collecting the unrelated information.

This inconvenient and troublesome process of collecting patient information may diminish the patient's motivation to continue using the method. As a result, a patient may discontinue using the healthcare system.

### SUMMARY

According to an aspect, a healthcare system is provided. The healthcare system includes a server configured to receive user information and obtain healthcare items based on the user information, a gateway configured to receive the healthcare items, and a vital information measurement sensor configured to measure vital information of a user.

The server may include a Clinical Decision Support system (CDSS).

The server may be configured to obtain the healthcare items using the CDSS.

The server may be at least one of a hospital server, a healthcare center server, a national health insurance corporation (NHIC) server, and a public domain server.

The healthcare center server may be at least one of a smart care center server, a pharmacy server, and a health center server.

The user information may include user diagnostic information created by a medical professional.

The server may include a database including at least one of personal information, diagnostic information, healthcare items, measured vital information, and a result of analyzing the measured vital information of the user.

The server may transmit the healthcare items to the gateway.

Communication between the server and the gateway may be performed using a method selected from at least one of a group including WiFi, wired/wireless LAN, CDMA, WiBro, 3G, 4G, Long Term Evolution (LTE), and a Public Switched Telephone Network (PSTN) communication.

In response to the healthcare items being corrected, the server may store the corrected healthcare items.

The server may transmit the corrected healthcare items to the gateway.

The healthcare items may include at least one of vital information to be measured by the user, the number of times that the vital information has been measured, a measurement time of the vital information, a method of measuring the vital information, a kind of a drug to be taken, the number of units of the drug that need to be taken, a time when the drug is taken, a method of taking the drug, a menu, and an exercise plan of the user.

The gateway may be a terminal used to access the healthcare system.

The gateway may receive the vital information of the user from the vital information measurement sensor and transmit the vital information to the server.

The gateway may be at least one of a gateway including a display, a set top box type gateway, a smart phone type gateway, a mobile phone type gateway, a WiBro terminal type gateway, a WiFi wireless router type gateway, a PC type gateway, an IPTV type gateway, and a gateway including a vital information measurement sensor and a medical device.

The gateway may include a care module that is configured to perform at least one of a method of using the vital information measurement sensor, a method of transmitting data, a video call, a telephone call, and a survey.

The vital information measurement sensor may include at least one of a blood pressure gauge, a blood sugar gauge, an electronic stethoscope, a peak flow meter, an exercise tester, a menu analyzer, a life pattern analyzer, a video camera, a weighting scale, a body composition analyzer, a medical imaging device, a urine analyzer, or any combination thereof.

The vital information measurement sensor may transmit the measured vital information of the user to the gateway.

Communication between the vital information measurement sensor and the gateway may be performed using at least one method selected from a group including Bluetooth, infrared communication, WiFi, wired/wireless LAN, serial, ZigBee, RF and Universal Serial Bus (USB) communication.

The gateway may be separate from the server.

The vital information may include blood pressure, blood sugar level, heart rate, or any combination thereof.

In another aspect, a healthcare method is provided. The healthcare method includes determining healthcare items, accessing a healthcare system, transmitting the healthcare items, and measuring vital information according to the healthcare items.

The healthcare system may include a gateway which is a terminal that is used to access the healthcare system, a vital information measurement sensor to measure the vital information of the user and to transmit the vital information to the gateway, and a server including a database.

The determining the healthcare items may include receiving user diagnostic information by the server, and obtaining the healthcare items based on the user diagnostic information.

The obtaining of the healthcare items based on the user diagnostic information may include obtaining healthcare items related to user's health using a Clinical Decision Support system (CDSS) at the server.

The determining the healthcare items may include checking whether a predetermined period has been reached, and obtaining healthcare items including a number of measurement items to measure user's overall health, in response to the predetermined period being reached.

The accessing the healthcare system may include accessing the gateway, transmitting user account information from the gateway to the server, comparing the user account information with information in the user database by the server, and allowing access to the healthcare system in response to the user account information matching the information in the user database.

The transmitting the healthcare items may include selecting the healthcare items of the user who accesses the healthcare system by the server, and transmitting the selected healthcare items to the gateway.

The measuring the vital information according to the healthcare items may include displaying the healthcare items by the gateway, and measuring the vital information of the user according to the healthcare items using the vital information measurement sensor.

The healthcare method may further include, in response to the vital information being measured according to the healthcare items, transmitting the vital information to the server, and correcting the healthcare items based on the vital information by the server.

The correcting the healthcare items based on the vital information may include selecting an item which is to be measured only once during a predetermined period from the healthcare items, determining whether the item which is to be measured only once is measured, and deleting the item which is to be measured only once from the healthcare items and correcting the healthcare items, once the item which is to be measured only once is measured.

The healthcare method may further include storing the corrected healthcare items in the server and transmitting the corrected healthcare items to the gateway in response to the user accessing the healthcare system after the storing operation, in response to the server correcting the healthcare items based on the vital information.

The transmitting of the corrected healthcare items and transmitting of the corrected healthcare items to the gateway in response to the user accessing the healthcare system after the storing operation may include transmitting the healthcare items including the item which needs to be measured only once in response to the user accessing the healthcare system after the predetermined period.

The healthcare method may further include in response to the vital information being measured according to the healthcare items, determining whether an error has occurred in the measurement of the vital information, and requesting re-measurement of the vital information upon determining that an error has occurred in measurement of the vital information.

The determining whether an error has occurred in measurement of the vital information may include determining whether the vital information is within a predetermined range, and determining that the error has occurred in the measurement of the vital information in response to the vital information not being within the predetermined range.

The requesting of the re-measurement of the vital information may include displaying information indicating that an error has occurred in measurement of the vital information, and displaying a recommendation that the vital information be re-measured.

The healthcare method may further include adding a re-measurement item of the vital information, in which the error has occurred, to the healthcare items in response to the re-measurement of the vital information not being performed.

In another aspect, there is provided a method of a terminal for use as a gateway in a remote healthcare system, the method including receiving, from a server, an instruction related to a patient's healthcare, determining healthcare items for a patient based on the received instruction, transmitting the information associated with the healthcare items to a sensor of the patient, receiving the requested healthcare items measured by the sensor, and transmitting the requested healthcare items to the server.

The server and the gateway may be connected via a wireless internet connection, and the sensor and the gateway may be connected via a wireless connection.

The server and the gateway may be connected via at least one of WiFi, wired/wireless LAN, CDMA, WiBro, 3G, 4G, Long Term Evolution (LTE), and Public Switched Telephone Network (PSTN) communication network, and the sensor and the gateway may be connected via at least one of Bluetooth communication, infrared communication, WiFi, wired/wireless LAN, ZigBee, RF, and a Universal Serial Bus (USB) communication.

Other features and aspects may be apparent from the following detailed description, the drawings, and the claims.

### BRIEF DESCRIPTION OF THE DRAWINGS

FIG. 1 is a diagram illustrating an example of a healthcare system; and

FIGS. 2 to 4 are flowcharts illustrating examples of a healthcare method.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. Also, descriptions of well-known functions and constructions may be omitted for increased clarity and conciseness.

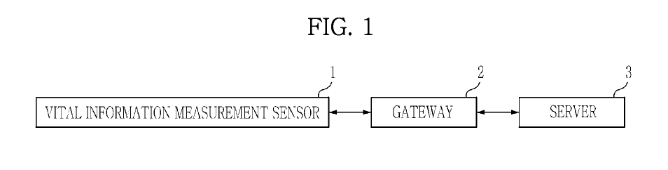

FIG. 1 illustrates an example of a healthcare system.

Referring to FIG. 1, the healthcare system includes a vital information measurement sensor 1, a gateway 2, and a server 3. The vital information measurement sensor 1 may measure vital information of a user. The server 3 may receive the vital information of the user and obtain healthcare items related to a user's disease or a user's health based on the vital information of the user. The gateway 2 may receive the healthcare items from the server 3.

The vital information measurement sensor 1 corresponds with a measurement device, which measures the vital information of the user. As a non-limiting example, the vital information measurement sensor 1 may include a blood pressure gauge, a blood sugar gauge, an electronic stethoscope, a peak flow meter, an exercise tester, a menu analyzer, a life pattern analyzer, a video camera, a weighing scale, a body composition analyzer, a medical imaging device, a urine analyzer, and the like. For example, the vital information may be biological information that is capable of being measured from a patient.

In response to vital information being measured using the vital information measurement sensor 1, the vital information measurement sensor 1 may transmit the measured vital information to the gateway 2.

The gateway 2 may include a communication unit (not shown) to perform communications, a controller (not shown) to generate information received from the communication unit, and a display unit (not shown) to display information related to a patient according to the control signal of the controller. The controller may generate a control signal based on the received information.

The gateway 2 corresponds to a user terminal which enables a user to access a healthcare service through a remote healthcare system. The gateway 2 may receive a healthcare service including healthcare items created based on user diagnostic information created by a medical professional through a server. The gateway 2 may provide the healthcare service to the user, for example, a patient. In response to the user measuring a variety of vital information using the vital information measurement sensor 1 according to the healthcare service, the gateway receives the measured vital information from the vital information measurement sensor 1, and the gateway 2 transmits the received vital information to the server, thereby enabling medical professionals to remotely treat the patient.

The user may use the gateway 2. For example, the user may be a patient who has a disease or a user who receives a remote healthcare service to monitor their health using the gateway 2 for healthcare.

As a non-limiting example, the gateway 2 may be a terminal, such as a computer, a tablet, a personal digital assistant, a mobile terminal, an appliance, a medical device, and the like. The gateway 2 may include a display, an IPTV type gateway, a cable TV type gateway, a set top box type gateway, a smart phone type gateway, a mobile phone type gateway, a WiBro terminal type gateway, a WiFi wireless router type gateway, a PC type gateway, a tablet PC, or a gateway including a medical device.

The healthcare items are created based on user diagnostic information by the medical professional, and refer to items for healthcare related to user's health.

As a non-limiting example, the healthcare items may include vital information of a user to be measured by a user at home, the number of times that vital information has been measured, a measurement time of the vital information, a method of measuring the vital information including a method of using the vital information measurement sensor, a drug to be taken by the user during healthcare, the number of units of the drug that are to be taken, a time when the drug is to be taken, a method of taking the drug, a user menu including a recommended food list and a restricted food list, an exercise plan for improving user's health, and the like.

The communication unit of the gateway 2 may receive the healthcare service from the server 3 and may receive an instruction and recommendation related to user healthcare from a medical professional through the server 3.

As a non-limiting example, communications between the server 3 and the communication unit of the gateway 2 may be performed using a WiFi, CDMA, wired/wireless LAN, WiBro, 3G, 4G, a Public Switched Telephone Network (PSTN) communication, and the like.

The communication unit of the gateway may receive the vital information measured using the vital information measurement sensor 1 according to the healthcare items and may transmit the vital information to the server 3.

As a non-limiting example, communications between the communication unit of the gateway 2 and the vital information measurement sensor 1 may be performed using Bluetooth, infrared communication, WiFi, wired/wireless LAN, ZigBee, Serial or Universal Serial Bus (USB) communication, and the like.

The display unit of the gateway may display healthcare information such as care information and user information. The display unit enables the user to view the information, in order to administer healthcare according to the healthcare items. The display unit may be a touchscreen display. The touchscreen display may have various input buttons displayed that may be directly touched to input external information. Accordingly, a screen may be changed according to the inputted external information. Although the touchscreen method is described, other implementations are understood.

As an example, the controller of the gateway may be a microcomputer configured to provide the user with care information according to the healthcare items through the display unit. The controller may administer healthcare according to the received healthcare items, and the controller may store personal information of a user, user's current health, healthcare items transmitted from the server, progress states of the healthcare items, measured vital information of the user transmitted from the vital information measurement sensor, a change in user's health through analysis of the measured vital information of the user, an additional information related to healthcare, and the like, which may be transmitted from the server.

The server 3 may include a user database for storing information about a user that uses the healthcare service. The server 3 may receive information related to healthcare, such as vital information of the user, from the gateway 2 and the server 3 may transmit the information to the medical professionals, thereby enabling the medical professionals to remotely administer user healthcare.

For example, the user database may include personal information of the user who uses the healthcare service, diagnostic information of the user created by the medical professional, healthcare items created based on the diagnostic information, measured vital information, a result of analyzing the measured vital information, and the like.

The server 3 may include various servers. For example, the server may include a hospital server, a care server which is operated by a smart care center or a healthcare center so as to connect a user and a hospital, a server operated by a pharmacy, a server operated by a health center, a server operated by a national health insurance corporation (NHIC), a public domain provided by a company, and the like.

Hereinafter, as a non-limiting example, functions of a server of a healthcare system including a hospital server and a care server operated by a smart care center or a healthcare center, are described.

The hospital server may receive user diagnostic information created by a medical professional, and the hospital server may transmit the user diagnostic information to the care server. In response to the user diagnostic information being transmitted from the hospital server to the care server, the care server may receive the user diagnostic information and obtain healthcare items related to a user's health based on the user diagnostic information.

The care server may transmit the obtained healthcare items to the hospital server. In response to the hospital server receiving the healthcare items from the care server, the hospital server may send the healthcare items to a medical professional. Thus, the hospital server may enable the medical professional to determine whether the healthcare items obtained by the care server are related to the user's health so as to address the healthcare items. In another example, the medical professional may correct the healthcare items to correspond with the healthcare items related to the user's health.

In response to the medical professional determining that the healthcare items are related to the user's health or correcting the healthcare items, the hospital server may transmit the healthcare items to the care server.

The care server may receive and store the healthcare items in the user database.

Hereinafter, an example of a remote healthcare method using the healthcare system including the vital information measurement sensor, the gateway, and the server is described.

The healthcare method using the healthcare system includes determining healthcare items related to the user's health at the server, enabling a user to access the healthcare system, transmitting the healthcare items related to the health of the user to the gateway, and measuring vital information of the user using the vital information measurement sensor according to the healthcare items.

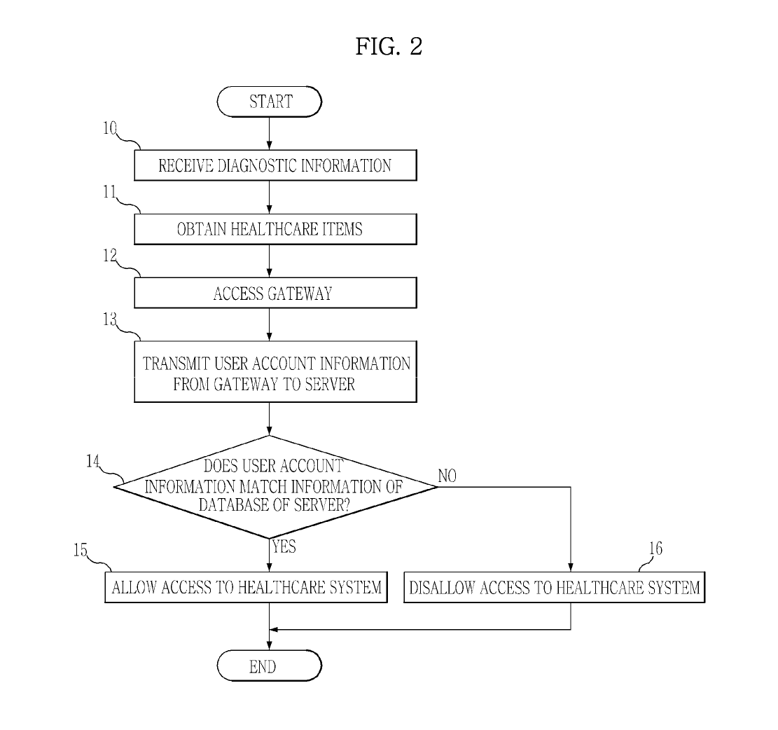

FIG. 2 illustrates an example of a healthcare method.

Referring to FIG. 2, the healthcare method includes determining healthcare items related to the user's health and enabling the user to access the healthcare system.

The server receives user diagnostic information created by a medical professional (10). The server obtains healthcare items related to the user's health based on the user diagnostic information (11), in response to receiving the user diagnostic information.

For example, the server may include a Clinical Decision Support System (CDSS) and one or more healthcare items may be obtained using the CDSS.

The CDSS is a set of programs to aid a medical professional to perform diagnosis or treatment by providing knowledge. The CDSS may analyze medical knowledge in association with clinical information such as diagnosis, operations, vital signs, prescription, and test results. Based on the analysis, the CDSS may make medical recommendations to the medical professional so as to improve accuracy and speed, minimize errors, and improve service quality.

The server may obtain healthcare items including items which may be measured by a user, in addition to identified healthcare items related to the user's health. For example, the server may obtain healthcare items including care items which may be measured by the user at home. The user may measure the user's health at predetermined intervals. For example, the measurement of the user's health may occur annually or bi-annually. The server may determine when to measure the user's health. The server may obtain healthcare items including measurement items to measure the user's overall health, in response to a determination that the predetermined period has been reached. Accordingly, the healthcare system may periodically provide a user with an overall healthcare service corresponding to a medical checkup of a hospital, in addition to the healthcare service related to the user's health.

The user may access the healthcare system in order to receive the healthcare service from the healthcare system. Referring to FIG. 2, the user accesses the gateway in order to access the healthcare system (12). For example, the gateway may be accessed by inputting user account information including an ID and password of a user into the gateway.

In response to the user accessing the gateway, the gateway transmits the user account information to the server (13). The server receives the user account information from the gateway, compares the user account information of the user with the user account information stored in the user database to check whether the user account information matches (14).

The server allows the user access to the healthcare system to the gateway (15), in response to the user account information matching. In response to access to the healthcare system being allowed, the user may use the healthcare service provided by the healthcare system.

The server denies the user access to the gateway (16), in response to the user account information not matching. The user may access the gateway again in order to gain access to the healthcare system. In response to the user accessing the healthcare system again, the above-described operations may be applied again.

In response to the user accessing the healthcare system, the gateway may receive the healthcare items from the server and provide the healthcare items to the user. The user may perform a scheduled healthcare procedure according to the healthcare items. For example, the user may measure vital information using the vital information measurement sensor 1 to check the user's health.

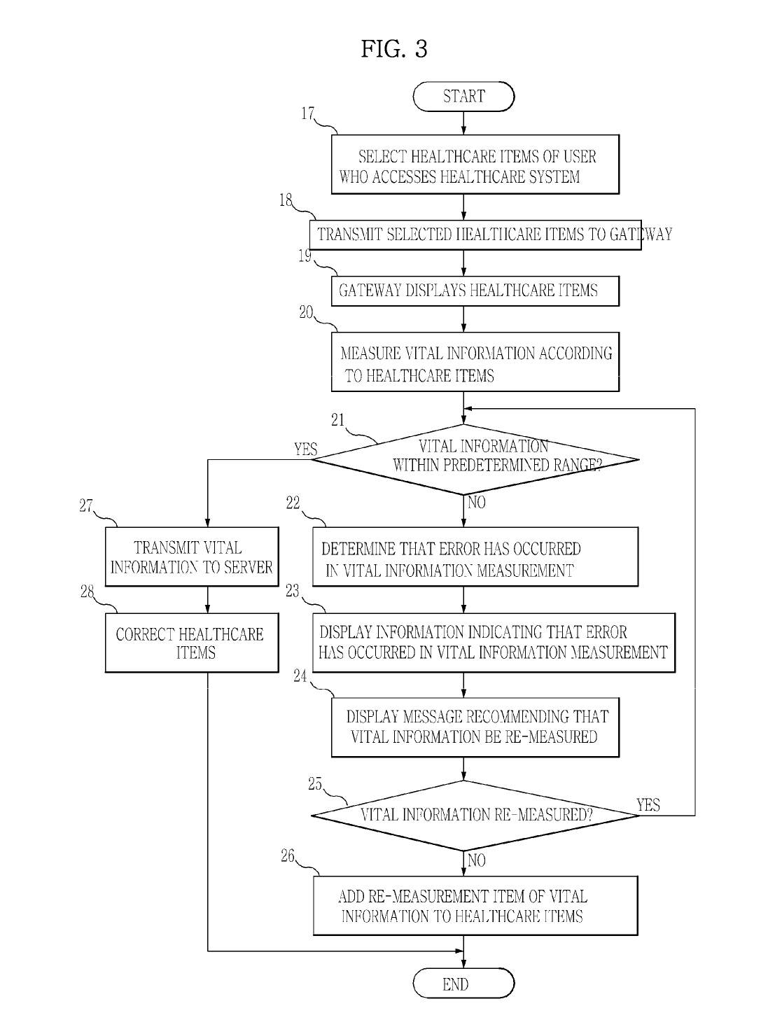

FIG. 3 illustrates another example of a healthcare method.

Referring to FIG. 3, the healthcare method includes transmitting the healthcare items related to the health of the user who accesses the healthcare system to the gateway and measuring the vital information of the user using the vital information measurement sensor 1.

In response to access to the healthcare system being allowed, the server selects the healthcare items of the user by comparing the information of the user accessing the healthcare system with the information of the user database of the server (17) and transmits the selected healthcare items to the gateway (18).

The gateway displays the healthcare items received from the server on the display unit so as to enable the user to identify which of the healthcare items are to be measured (19). The gateway includes a care module to provide a method of using one or more various vital information measurement sensors, a method of transmitting data including a variety of measured data and a method of placing a call with the medical professional or to conduct a survey including a user health questionnaire or user satisfaction for a service used through the healthcare system, in order to enable the user to perform a healthcare procedure. The call may be a video call or a telephone call.

The user may measure the vital information using the vital information measurement sensor according to the selected healthcare items (20).

In response to the vital information measurement of the user being completed, a determination is made whether the vital information measurement is successfully performed. In other words, a determination is made whether an error has occurred in the vital information measurement. The determination may be made based on the vital information measurement information, which the gateway receives from the vital information measurement sensor, or the medical professional receives through the server.

In response to the determination that an error has or has not occurred in the vital information measurement being made by the vital information measurement sensor or the gateway, the vital information measurement sensor or the gateway determines whether or not the measured vital information is within a predetermined range (21).

As an example, the predetermined range may be a range from a minimum value to a maximum value of the vital information, and the minimum value and the maximum value of the vital information may be a minimum value and a maximum value of a sum of vital information value ranges when a user is healthy or sick.

The vital information value outside of the predetermined range corresponds to the vital information value which may not be obtained. Therefore, in response to the vital information being outside of the predetermined range, the vital information measurement sensor or the gateway determines that an error has occurred in vital information measurement (22).

In response to a determination that an error has occurred in vital information measurement, information indicating that an error has occurred in vital information measurement is displayed (23). The information may be displayed in the vital information measurement sensor or the gateway. The information indicating that an error has occurred in the vital information measurement may be announced to the user using a sound or a voice signal, or by displaying a message indicating that an error has occurred in vital information measurement on the display unit of the gateway. It is understood that the announcement of the error is not limited to the above examples.

In response to the information indicating that an error has occurred in vital information measurement being displayed, a message recommending that the vital information be re-measured is displayed (24). As a non-limiting example, the message may be announced to the user using a voice signal or may be displayed on the display unit of the gateway.

If the vital information is re-measured and is in the predetermined range, the vital information is transmitted to the server (27). The vital information may be transmitted to the server by transmitting the vital information from the vital information measurement sensor to the gateway and transmitting the vital information from the gateway to the server.

The server receives the vital information and corrects the user healthcare items based on the received vital information (28). The correcting operation includes the server analyzing the vital information so as to check a change in user's health, performing correction such as adding of a new measurement item or removal of an unrelated measurement item according to the change, and obtaining healthcare items related to the user's health changed after vital information measurement.

The server may transmit the healthcare items, which are corrected to relate to the user's current health, to the gateway in response to the user accessing the healthcare system later and enabling the user to receive the healthcare service best related to the user's health according to the corrected healthcare items.

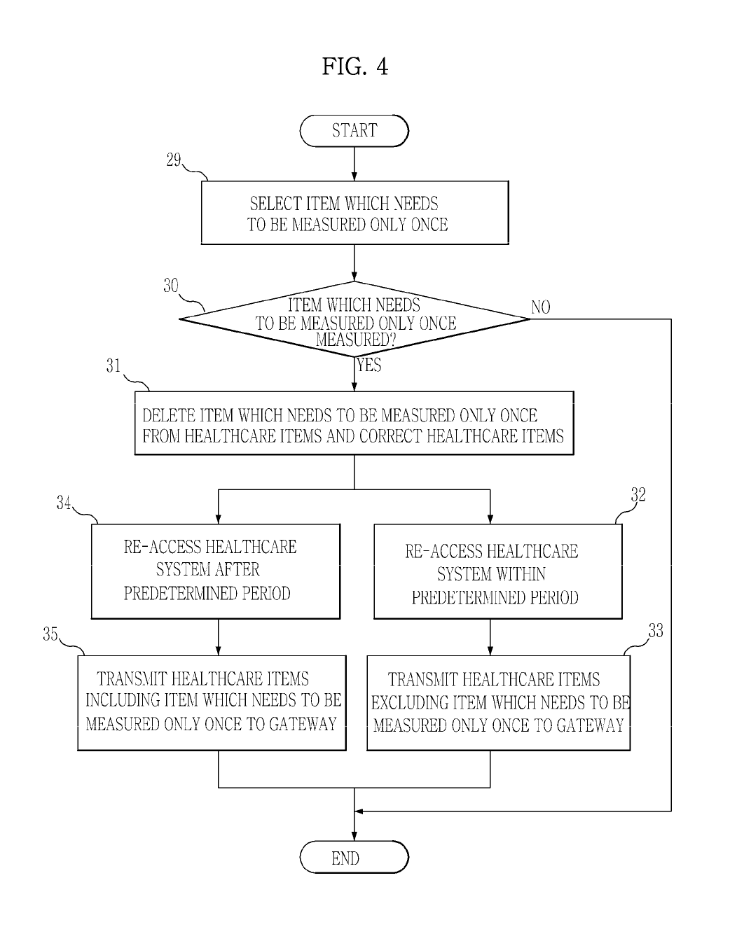

An example of correcting the healthcare items based on the vital information at the server is described with reference to FIG. 4.

In response to an item needing to be measured only once during the predetermined period (for example, a day or a week) being included in the healthcare items, the server checks whether or not the item which needs to be measured only once is measured using the received vital information of the user (29 and 30). In response to a determination that an item which needs to be measured only once has been measured, the server deletes the item which needs to be measured only once from the healthcare items and corrects the healthcare items (31). In response to the user re-accessing the healthcare system within a predetermined period (32), the healthcare items excluding the item which needs to be measured only once may be transmitted to the gateway and provided to the user (33).

In response to the healthcare items being corrected, the healthcare items may be provided excluding the item which needs to be measured only once to the user when the user re-accesses the healthcare system during the predetermined period and prevent the user from repeating unrelated measurements. Accordingly, the user may efficiently receive the healthcare service using the healthcare items without adding unrelated care items.

In response to the user re-accessing the healthcare system after the predetermined period (34), the server includes the item which needs to be measured only once in the healthcare items and transmits the healthcare items to the gateway so that the item which needs to be measured only once is measured (35). In response to an item which needs to be measured once every day or every week being included in the healthcare items, performing accurate measurement is possible without repetition by correcting the healthcare items using the above method.

During a determination of whether or not the vital information is re-measured according to the request for re-measurement of the vital information, in response to the vital information of the user not being re-measured, a re-measurement item of the vital information in which an error in the measurement has occurred is added to the healthcare items (26). That is, in response to the user ignoring the recommendation for the re-measurement of the vital information or not confirming the recommendation such that the vital information is not re-measured, the gateway adds the re-measurement item of the vital information to the healthcare items and stores the healthcare items or the gateway transmits information indicating that the vital information is not re-measured to the server such that the server adds and stores the re-measurement item of the vital information to the healthcare items of the user database.

By adding the re-measurement item of the vital information to the healthcare items, in response to the healthcare system being completed without re-measuring the vital information, the healthcare system enables the user to re-measure the vital information in response to the user accessing the healthcare system later. Thereby, a more accurate healthcare of the user may be achieved.

According to the examples, minimization of user inconvenience or troublesomeness is possible when obtaining vital information, by providing the healthcare items related to a user's disease or health.

In addition, efficient use of a healthcare service is possible by providing the healthcare items related to a user's disease or health.

A home healthcare system may include the healthcare system.

Program instructions to perform a method described herein, or one or more operations thereof, may be recorded, stored, or fixed in one or more computer-readable storage media. The program instructions may be implemented by a computer. For example, the computer may cause a processor to execute the program instructions. The media may include, alone or in combination with the program instructions, data files, data structures, and the like. Examples of computer-readable storage media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as produced by a compiler, and files containing higher level code that may be executed by the computer using an interpreter. The program instructions, that is, software, may be distributed over network coupled computer systems so that the software is stored and executed in a distributed fashion. For example, the software and data may be stored by one or more computer readable storage mediums. Also, functional programs, codes, and code segments for accomplishing the example embodiments disclosed herein can be easily construed by programmers skilled in the art to which the embodiments pertain based on and using the flow diagrams and block diagrams of the figures and their corresponding descriptions as provided herein. Also, the described unit to perform an operation or a method may be hardware, software, or some combination of hardware and software. For example, the unit may be a software package running on a computer or the computer on which that software is running.

As a non-exhaustive illustration only, a terminal/device/unit described herein may refer to mobile devices such as a cellular phone, a personal digital assistant (PDA), a digital camera, a portable game console, and an MP3 player, a portable/personal multimedia player (PMP), a handheld e-book, a portable lab-top PC, a global positioning system (GPS) navigation, a tablet, a sensor, and devices such as a desktop PC, a high definition television (HDTV), an optical disc player, a setup box, a home appliance, and the like that are capable of wireless communication or network communication consistent with that which is disclosed herein.

A computing system or a computer may include a microprocessor that is electrically connected with a bus, a user interface, and a memory controller. It may further include a flash memory device. The flash memory device may store N-bit data via the memory controller. The N-bit data is processed or will be processed by the microprocessor and N may be 1 or an integer greater than 1. Where the computing system or computer is a mobile apparatus, a battery may be additionally provided to supply operation voltage of the computing system or computer. It will be apparent to those of ordinary skill in the art that the computing system or computer may further include an application chipset, a camera image processor (CIS), a mobile Dynamic Random Access Memory (DRAM), and the like. The memory controller and the flash memory device may constitute a solid state drive/disk (SSD) that uses a non-volatile memory to store data.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A healthcare system comprising:
a server configured to receive user information and obtain healthcare items based on the user information;
a gateway configured to receive the healthcare items; and
a vital information measurement sensor configured to measure vital information of a user.

2. The healthcare system according to claim 1, wherein the server comprises a Clinical Decision Support system (CDSS).

3. The healthcare system according to claim 2, wherein the server is configured to obtain the healthcare items using the CDSS.

4. The healthcare system according to claim 1, wherein the server is at least one of a hospital server, a healthcare center server, a national health insurance corporation (NHIC) server, and a public domain server.

5. The healthcare system according to claim 4, wherein the healthcare center server is at least one of a smart care center server, a pharmacy server, and a health center server.

6. The healthcare system according to claim 1, wherein the user information comprises user diagnostic information created by a medical professional.

7. The healthcare system according to claim 1, wherein the server comprises a database including at least one of personal information, diagnostic information, healthcare items, measured vital information, and a result of analyzing the measured vital information of the user.

8. The healthcare system according to claim 1, wherein the server is further configured to transmit the healthcare items to the gateway.

9. The healthcare system according to claim 1, wherein communication between the server and the gateway is performed using a method selected from at least one of WiFi, wired/wireless LAN, CDMA, WiBro, 3G, 4G, Long Term Evolution (LTE), and Public Switched Telephone Network (PSTN) communication.

10. The healthcare system according to claim 1, wherein, in response to the healthcare items being corrected, the server is further configured to store the corrected healthcare items.

11. The healthcare system according to claim 10, wherein the server is further configured to transmit the corrected healthcare items to the gateway.

12. The healthcare system according to claim 1, wherein the healthcare items comprise at least one of vital information to be measured by the user, the number of times that the vital information has been measured, a measurement time of the vital information, a method of measuring the vital information, a kind of a drug to be taken, the number of units of the drug that need to be taken, a time when the drug is taken, a method of taking the drug, a menu, and an exercise plan of the user.

13. The healthcare system according to claim 1, wherein the gateway is a terminal used to access the healthcare system.

14. The healthcare system according to claim 1, wherein the gateway is configured to receive the vital information of the user from the vital information measurement sensor and transmit the vital information to the server.

15. The healthcare system according to claim 1, wherein the gateway is at least one of a gateway including a display, a set top box type gateway, a smart phone type gateway, a mobile phone type gateway, a WiBro terminal type gateway, a WiFi wireless router type gateway, a PC type gateway, an IPTV type gateway, and a gateway including a vital information measurement sensor and a medical device.

16. The healthcare system according to claim 1, wherein the gateway comprises a care module that is configured to perform at least one of a method of using the vital information measurement sensor, a method of transmitting data, a video call, a telephone call, and a survey.

17. The healthcare system according to claim 1, wherein the vital information measurement sensor comprises at least one of a blood pressure gauge, a blood sugar gauge, an electronic stethoscope, a peak flow meter, an exercise tester, a menu analyzer, a life pattern analyzer, a video camera, a weighting scale, a body composition analyzer, a medical imaging device, and a urine analyzer.

18. The healthcare system according to claim 1, wherein the vital information measurement sensor is configured to transmit the measured vital information of the user to the gateway.

19. The healthcare system according to claim 1, wherein communication between the vital information measurement sensor and the gateway is performed using at least one of Bluetooth, infrared communication, WiFi, wired/wireless LAN, serial, ZigBee, RF, and Universal Serial Bus (USB) communication.

20. A healthcare method comprising:
determining healthcare items;
accessing a healthcare system;
transmitting the healthcare items; and
measuring vital information according to the healthcare items.

21. The healthcare method according to claim 20, wherein the healthcare system comprises:
a gateway which is a terminal that is used to access the healthcare system;
a vital information measurement sensor to measure the vital information of the user and to transmit the vital information to the gateway; and
a server including a database.

22. The healthcare method according to claim 21, wherein the determining the healthcare items comprises:
receiving user diagnostic information by the server; and
obtaining the healthcare items based on the user diagnostic information.

23. The healthcare method according to claim 22, wherein the obtaining of the healthcare items based on the user diagnostic information comprises obtaining healthcare items related to user's health using a Clinical Decision Support system (CDSS) at the server.

24. The healthcare method according to claim 21, wherein the determining the healthcare items comprises:
checking whether a predetermined period has been reached; and
obtaining healthcare items including a number of measurement items to measure a user's overall health, in response to the predetermined period being reached.

25. The healthcare method according to claim 21, wherein the accessing the healthcare system comprises:
accessing the gateway;
transmitting user account information from the gateway to the server;
comparing the user account information with information in the user database by the server; and
allowing access to the healthcare system, in response to the user account information matching the information in the user database.

26. The healthcare method according to claim 21, wherein the transmitting the healthcare items comprises:
selecting the healthcare items of the user who accesses the healthcare system by the server; and
transmitting the selected healthcare items to the gateway.

27. The healthcare method according to claim 21, wherein the measuring the vital information according to the healthcare items comprises:
displaying the healthcare items by the gateway; and
measuring the vital information of the user according to the healthcare items using the vital information measurement sensor.

28. The healthcare method according to claim 21, further comprising:
in response to the vital information being measured according to the healthcare items,
transmitting the vital information to the server; and
correcting the healthcare items based on the vital information by the server.

29. The healthcare method according to claim 28, wherein the correcting the healthcare items based on the vital information comprises:
selecting an item which is to be measured only once during a predetermined period from the healthcare items;
determining whether the item which is to be measured only once has been measured; and
deleting the item which is to be measured only once from the healthcare items and correcting the healthcare items, once the item which is to be measured only once has been measured.

30. The healthcare method according to claim 29, further comprising the server storing the corrected healthcare items and transmitting the corrected healthcare items to the gateway when the user accesses the healthcare system later, in response to the server correcting the healthcare items based on the vital information.

31. The healthcare method according to claim 30, wherein the transmitting the corrected healthcare items when the user accesses the healthcare system includes transmitting the healthcare items including the item which is to be measured only once when the user accesses the healthcare system after the predetermined period.

32. The healthcare method according to claim 21, further comprising:
in response to the vital information being measured according to the healthcare items,
determining whether an error has occurred in the measurement of the vital information; and
requesting re-measurement of the vital information upon determining that an error has occurred in measurement of the vital information.

33. The healthcare method according to claim 32, wherein the determining whether an error has occurred in measurement of the vital information comprises:
determining whether the vital information is within a predetermined range; and
determining that the error has occurred in the measurement of the vital information, in response to the vital information not being within the predetermined range.

34. The healthcare method according to claim 32, wherein the requesting of the re-measurement of the vital information comprises:
displaying information indicating that an error has occurred in measurement of the vital information; and
displaying a recommendation that the vital information be re-measured.

35. The healthcare method according to claim 32, further comprising adding a re-measurement item of the vital information, in which the error has occurred, to the healthcare items, in response to the re-measurement of the vital information not being performed.

36. The healthcare system according to claim 1, wherein the gateway is separate from the server.

37. The healthcare system according to claim 1, wherein the vital information includes blood pressure, blood sugar level, heart rate, or any combination thereof.

38. A method of a terminal for use as a gateway in a remote healthcare system, the method comprising:
receiving, from a server, an instruction related to a patient's healthcare;
determining healthcare items for a patient based on the received instruction;
transmitting the information associated with the healthcare items to a sensor of the patient;
receiving the requested healthcare items measured by the sensor; and
transmitting the requested healthcare items to the server.

39. The method of claim 38, wherein the server and the gateway are connected via a wireless internet connection, and the sensor and the gateway are connected via a wireless connection.

40. The method of claim 39, wherein the server and the gateway are connected via at least one of WiFi, wired/wireless LAN, CDMA, WiBro, 3G, 4G, Long Term Evolution (LTE), and Public Switched Telephone Network (PSTN) communication network, and the sensor and the gateway are connected via at least one of Bluetooth communication, infrared communication, WiFi, wired/wireless LAN, ZigBee, RF, and a Universal Serial Bus (USB) communication.
